(19) United States
(12) Patent Application Publication
(10) Pub. No.: US 2014/0088368 A1
(43) Pub. Date: Mar. 27, 2014

(54) SURGICAL INSTRUMENT, SURGICAL MESH AND SURGICAL RETRACTION MEANS OF THE INSTRUMENT, AND SURGICAL METHOD USING THE INSTRUMENT

(71) Applicant: Kwang-Tai Park, Seoul (KR)

(72) Inventor: Kwang-Tai Park, Seoul (KR)

(21) Appl. No.: 13/978,377

(22) PCT Filed: Jan. 17, 2013
(86) PCT No.: PCT/KR2013/000379

§ 371 Date: Jul. 3, 2013

(30) Foreign Application Priority Data
Jan. 18, 2012 (KR) 2012-0005915

(51) Int. Cl. A61B 17/02

(52) U.S. Cl. CPC A61B 17/0218
USPC 600/204

(57) ABSTRACT

A surgical instrument, a surgical mesh and a surgical retraction means of the instrument, and a surgical method using the instrument, in which the surgical retraction means is connected to the mesh and to a body tissue at opposite ends, and so the tissue retraction force can be distributed to several holding parts that fasten the mesh to the inner surface of a body cavity, thereby efficiently drawing back the body tissue in various directions. The surgical instrument includes a mesh fastened in an open state to the inner surface of the body cavity using a fastening means; and a surgical retraction means selectively and removably connected at a first end to a point of the mesh and connected at a second end to a part of the internal tissue that is required to be drawn back, so that the surgical retraction means can efficiently draw back the internal tissue.

### CROSS REFERENCE RELATED APPLICATION

This application claims foreign priority of Korean Patent Application No. 2012-0005915, filed on Jan. 18, 2012, which is incorporated by reference in their entirety into this application.

### Technical Field

The present invention relates, in general, to a surgical instrument, a surgical mesh and a surgical retraction means of the instrument, and a surgical method using the instrument and, more particularly, to a surgical instrument, a surgical mesh and a surgical retraction means of the instrument, and a surgical method using the instrument, in which a first end of the surgical retraction means is connected to the mesh and a second end thereof is connected to a body tissue, and so the tissue retraction force of the surgical retraction means can be distributed to a plurality of holding parts that fasten the mesh to the inner surface of a body cavity, thereby easily and efficiently drawing back the body tissue in various directions.

### Background Art

Generally, laparoscopic surgery is a surgical method in which a small-sized incision that is 0.5˜1.5 cm in size is formed in the abdomen and an operative procedure is performed by inserting a video camera and various instruments into the abdomen through the incision.

The laparoscopic surgery is advantageous in that it is cosmetically preferable to a patient due to its resulting in only a small-sized surgical wound, it can greatly reduce pain caused by the incision, and it can allow the patient to recuperate quickly and return to his or her daily life quickly. Accordingly, laparoscopic surgery is widely performed these days.

During the above-mentioned laparoscopic surgery, it is required to adequately expose a body part that requires a medical procedure or a medical treatment. Therefore, retraction treatments for a variety of body tissues and internal organs are required to be performed during laparoscopic surgery.

For example, as shown in FIG. 1, to perform a surgical treatment, such as an incision or excision of a specified body part P1, a retraction treatment for a tissue T that is related to the specified part is required.

Accordingly, to successively perform laparoscopic surgery, a surgeon who takes charge of the surgical treatment and another surgeon or an assistant who takes charge of the retraction treatment should participate in the laparoscopic surgery.

For example, a surgical treatment applier AP1 that is handled by the surgeon in charge of the surgical treatment may be inserted into a body cavity through a port PT1, and retraction treatment appliers AP2, AP3 that are handled by the surgeon in charge of the retraction treatment may be inserted into the body cavity through respective ports PT2, PT3.

Particularly, when several parts require the retraction treatment, many surgeons or assistants should participate in the laparoscopic surgery.

To perform laparoscopic surgery, at least two experts should participate in the surgery as described above, so the laparoscopic surgery cannot be performed in a one-man hospital that is managed by one surgeon.

Further, to perform laparoscopic surgery, it is required to form a plurality of ports for various treatments (for example, the port PT1 for surgical treatment, such as an incision, an excision, etc., of a specified part, and the ports PT2, PT3 for retraction treatment of another part related to the specified part requiring the surgical treatment).

However, the forming of the plurality of ports in the body is problematic in that it is contrary to the cosmetic merits of laparoscopic surgery, that is, that laparoscopic surgery results in only small-sized surgical wound.

Further, another problem of the forming of the plurality of ports resides in that it increases the cost of the laparoscopic surgery.

Further, the above-mentioned forming of the plurality of ports is also problematic in that the damage caused to the body increases in proportion to the number of added ports.

Further, the conventional retraction method used in the laparoscopic surgery is problematic in that it is difficult to draw back the tissue in a variety of directions.

### DISCLOSURE

### Technical Problem

Accordingly, the present invention has been made keeping in mind the above problems occurring in the related art, and is intended to provide a surgical instrument, a surgical mesh and a surgical retraction means of the instrument, and a surgical method using the instrument, in which a first end of the surgical retraction means is connected to the mesh and a second end thereof is connected to a body tissue, and so the tissue retraction force of the surgical retraction means can be distributed to a plurality of holding parts that fasten the mesh to the inner surface of a body cavity, thereby easily and efficiently drawing back the body tissue in various directions.

### Technical Solution

In order to accomplish the above-mentioned object, in an aspect, the present invention provides a surgical instrument configured to be inserted and installed in a body cavity so as to draw back an internal tissue, the surgical instrument including: a mesh configured to be fastened in an open state to an inner surface of the body cavity using a fastening means; and a surgical retraction means configured to be selectively and removably connected at a first end thereof to a predetermined point of the mesh and configured to be connected at a second end thereof to a predetermined part of the internal tissue that is required to be drawn back, so that the surgical retraction means can draw back the internal tissue.

Preferably, the surgical retraction means includes: a mesh holding anchor configured to be selectively connected to the predetermined point of the mesh; a tissue holding anchor configured to be connected to the predetermined part of the internal tissue that is required to be drawn back; and a retraction string connected at a first end thereof to the mesh holding anchor and connected at a second end thereof to the tissue holding anchor.

Preferably, a thread that constitutes the mesh is provided on a surface thereof with a plurality of grooves for firmly connecting the surgical retraction means to the mesh.

Preferably, the fastening means includes at least one staple that can fasten a predetermined part of the mesh to a predetermined part of the inner surface of the body cavity by stapling them together.

Preferably, the fastening means includes at least one fastening string that can fasten the mesh to the inner surface of the body cavity by sewing with a needle in which the fastening string passes through predetermined openings of the mesh and through predetermined parts of the inner surface of the body cavity.

Preferably, the needle is configured as a straight needle or a curved needle.

Preferably, the mesh holding anchor or the tissue holding anchor is configured as any one of an open hook type anchor, a snap hook type anchor, a carabiner type anchor and a tongs type anchor.

Preferably, the tissue holding anchor is configured as the tongs type anchor provided with a tongs opening degree main control part, with a tongs opening degree sub-control part being formed on at least one side surface of the tongs opening degree main control part.

Preferably, the mesh holding anchor or the tissue holding anchor is provided with a string connection part that is configured as any one of a ring type, an open hook type, a snap hook type and a carabiner type, thereby being connected to the retraction string.

Preferably, the retraction string is configured to have elasticity.

Preferably, the retraction string is provided with a length control means.

Preferably, the surgical instrument further include: an applier including a shaft, a predetermined part of which is inserted into the body cavity through a port, a manipulation part provided in a first end of the shaft, and a handle part provided in a second end of the shaft so as to control the manipulation part, so that the applier can manipulate the mesh and the surgical retraction means that are inserted in the body cavity.

Preferably, the shaft is provided in the predetermined part thereof with a joint that can be controlled by the handle part so that a manipulation range of the manipulation part can be changed by the joint.

In order to accomplish the above-mentioned object, in another aspect, the present invention provides a surgical mesh configured to be inserted and installed in a body cavity so as to draw back an internal tissue.

Preferably, a thread that constitutes the mesh is provided on a surface thereof with a plurality of grooves for firmly connecting a surgical retraction means to the mesh.

In order to accomplish the above-mentioned object, in a further aspect, the present invention provides a surgical retraction means configured to be inserted into a body cavity so as to draw back an internal tissue, the surgical retraction means including: a mesh holding anchor; a tissue holding anchor; and a retraction string connected at a first end thereof to the mesh holding anchor and connected at a second end thereof to the tissue holding anchor.

Preferably, the mesh holding anchor or the tissue holding anchor is configured as any one of an open hook type anchor, a snap hook type anchor, a carabiner type anchor and a tongs type anchor.

Preferably, the tissue holding anchor is configured as the tongs type anchor provided with a tongs opening degree main control part, with a tongs opening degree sub-control part being formed on at least one side surface of the tongs opening degree main control part.

Preferably, the mesh holding anchor or the tissue holding anchor is provided with a string connection part that is configured as any one of a ring type, an open hook type, a snap hook type and a carabiner type, thereby being connected to the retraction string.

Preferably, the retraction string is configured to have elasticity.

Preferably, the retraction string is provided with a length control means.

In order to accomplish the above-mentioned object, in still another aspect, the present invention provides a surgical method performed in a state in which a surgical instrument is inserted into a body cavity through a port and draws back an internal tissue, the surgical method including: fastening a mesh in an open state to an inner surface of the body cavity using a fastening means; and selectively and removably connecting a first end of a surgical retraction means to a predetermined point of the mesh and connecting a second end of the surgical retraction means to a predetermined part of the internal tissue that is required to be drawn back, thereby drawing back the internal tissue.

Preferably, the surgical method further include: moving and connecting the first end of the surgical retraction means to another point of the mesh.

Preferably, the surgical retraction means includes: a mesh holding anchor selectively connected to the predetermined point of the mesh; a tissue holding anchor connected to the predetermined part of the internal tissue that is required to be drawn back; and a retraction string connected at a first end thereof to the mesh holding anchor and connected at a second end thereof to the tissue holding anchor, thereby drawing back the internal tissue.

Preferably, the surgical method further include: controlling a length of the retraction string.

Preferably, the moving and connecting the first end of the surgical retraction means to the other point of the mesh is performed by moving a connecting position of the mesh holding anchor to the other point of the mesh and by connecting the mesh holding anchor to the other point of the mesh.

### Advantageous Effects

As described above, the present invention is advantageous in that the first end of the surgical retraction means is connected to the mesh and the second end thereof is connected to the body tissue, and so the tissue retraction force of the surgical retraction means can be distributed to the plurality of holding parts that fasten the mesh to the inner surface of the body cavity, thereby easily and efficiently drawing back the body tissue in various directions.

Further, the surgical retraction means is configured such that the first end thereof can be selectively and removably connected to a point of the mesh, and so the present invention is advantageous in that, even when the first end of the surgical retraction means is repeatedly moved to other points of the mesh at various times, the surgical retraction means can minimize the damage caused to the inner surface of the body cavity.

Further, the present invention is advantageous in that it can allow a surgery to be optimally performed with a combination of optimum anchors suitable for a surgical environment by selecting the anchors from various types of mesh holding anchors and tissue holding anchors.

Further, when connecting a mesh holding anchor to the mesh, the mesh holding anchor can be fastened to one of the grooves that are formed on the surface of a thread constituting the mesh, and so the present invention is advantageous in that it can prevent undesired displacement of the mesh holding anchor.

Further, the mesh can be fastened to the inner surface of the body cavity using staples or fastening strings, and so the present invention is advantageous in that the fastening of the mesh scarcely forms a surgical wound.

Further, the present invention is advantageous in that it is possible to control the tissue gripping direction of the tissue holding anchor using the tongs opening degree sub-control part that is provided on at least one side surface of the tongs opening degree main control part of the tongs type tissue holding anchor.

Further, the retraction string that connects the mesh holding anchor and the tissue holding anchor to each other is configured to have elasticity, and so the present invention is advantageous in that it can continuously optimize the tissue retraction state even when the distance between the mesh holding anchor and the tissue holding anchor varies.

Further, the retraction string has the length control means, and so the present invention is advantageous in that it can optimize the tissue retraction state by controlling the length of the retraction string according to a variation in the distance between the mesh holding anchor and the tissue holding anchor.

Further, the present invention is advantageous in that it is possible to change using an applier having a joint the manipulation range of the manipulation part that is used to manipulate both the mesh and the surgical retraction means.

### DESCRIPTION OF DRAWINGS

FIG. 1 is a perspective view illustrating a retracted state of a body part that is required to be drawn back during a conventional laparoscopic surgery;

FIG. 2 is a perspective view illustrating a retracted state of a body part that is drawn back using a laparoscopic surgical instrument according to an embodiment of the present invention;

FIG. 3a is an enlarged view of a portion “A” of FIG. 2, which is a perspective view illustrating a state before an excision of tissue;

FIG. 3b is an enlarged view of the portion “A” of FIG. 2, which is a perspective view illustrating a state after the excision of tissue;

FIG. 3c is an enlarged view of the portion “A” of FIG. 2, which is a perspective view illustrating a modified connection state of a mesh holding anchor and a tissue holding anchor;

FIG. 3d is an enlarged view of the portion “A” of FIG. 2, which is a perspective view illustrating a further modified connection state of the mesh holding anchor and the tissue holding anchor;

FIG. 4 is a perspective view illustrating a state in which a mesh of the laparoscopic surgical instrument according to the embodiment of the present invention is fastened using staples;

FIG. 5a is a perspective view illustrating a state in which the mesh of the laparoscopic surgical instrument according to the embodiment of the present invention is fastened to an inner surface of the interior wall of the body using fastening strings;

FIG. 5b is a perspective view illustrating a state in which the mesh of the laparoscopic surgical instrument according to the embodiment of the present invention is fastened to an inner surface of the exterior wall of the body using the fastening strings;

FIG. 5c is a perspective view illustrating another embodiment of the fastening of the mesh of the laparoscopic surgical instrument of the present invention to the inner surface of the exterior wall of the body using the fastening strings;

FIG. 6a is a perspective view illustrating a straight needle that is used to fasten the mesh of the laparoscopic surgical instrument according to the embodiment of the present invention using a fastening string;

FIG. 6b is a perspective view illustrating a curved needle that is used to fasten the mesh of the laparoscopic surgical instrument according to the embodiment of the present invention using a fastening string;

FIG. 7a is a perspective view illustrating an open hook type anchor of the laparoscopic surgical instrument according to the embodiment of the present invention;

FIG. 7b is a perspective view illustrating a modification of the open hook type anchor of the laparoscopic surgical instrument according to the embodiment of the present invention;

FIG. 7c is a perspective view illustrating a snap hook type anchor of the laparoscopic surgical instrument according to the embodiment of the present invention;

FIG. 7d is a perspective view illustrating a carabiner type anchor of the laparoscopic surgical instrument according to the embodiment of the present invention;

FIG. 7e is a perspective view illustrating a tongs type anchor of the laparoscopic surgical instrument according to the embodiment of the present invention;

FIG. 7f is a perspective view illustrating a bulldog clip tongs type anchor of the laparoscopic surgical instrument according to an embodiment of the present invention;

FIG. 8 is a view schematically illustrating different kinds of string connection parts of the open hook type anchor of the laparoscopic surgical instrument according to the embodiment of the present invention;

FIG. 9 is a view schematically illustrating different kinds of string connection parts of the modified open hook type anchor of the laparoscopic surgical instrument according to the embodiment of the present invention;

FIG. 10 is a view schematically illustrating different kinds of string connection parts of the snap hook type anchor of the laparoscopic surgical instrument according to the embodiment of the present invention;

FIG. 11 is a view schematically illustrating different kinds of string connection parts of the carabiner type anchor of the laparoscopic surgical instrument according to the embodiment of the present invention;

FIG. 12 is a view schematically illustrating different kinds of string connection parts of the tongs type anchor of the laparoscopic surgical instrument according to the embodiment of the present invention;

FIG. 13 is a perspective view illustrating a straight applier of the laparoscopic surgical instrument according to the embodiment of the present invention;

FIG. 14 is a perspective view illustrating an applier having a joint of the laparoscopic surgical instrument according to the embodiment of the present invention;

FIG. 15a is a view schematically illustrating a straight bulldog clip tongs type anchor of the laparoscopic surgical instrument according to the embodiment of the present invention;

FIG. 15b is a view schematically illustrating a straight bulldog clip tongs type anchor having a tongs opening degree sub-control part of the laparoscopic surgical instrument according to the embodiment of the present invention;

FIG. 15c is a view schematically illustrating a curved bulldog clip tongs type anchor of the laparoscopic surgical instrument according to the embodiment of the present invention;

FIG. 15d is a view schematically illustrating a curved bulldog clip tongs type anchor having a tongs opening degree sub-control part of the laparoscopic surgical instrument according to the embodiment of the present invention;

FIGS. 16a and 16b are views schematically illustrating states in which the tongs opening degree sub-control part of the tongs type anchor are gripped by the straight applier of the laparoscopic surgical instrument according to the embodiment of the present invention;

FIG. 17 is a perspective view illustrating an example of conventional surgical staplers; and

FIG. 18 is a flowchart of a surgical method according to the embodiment of the present invention.

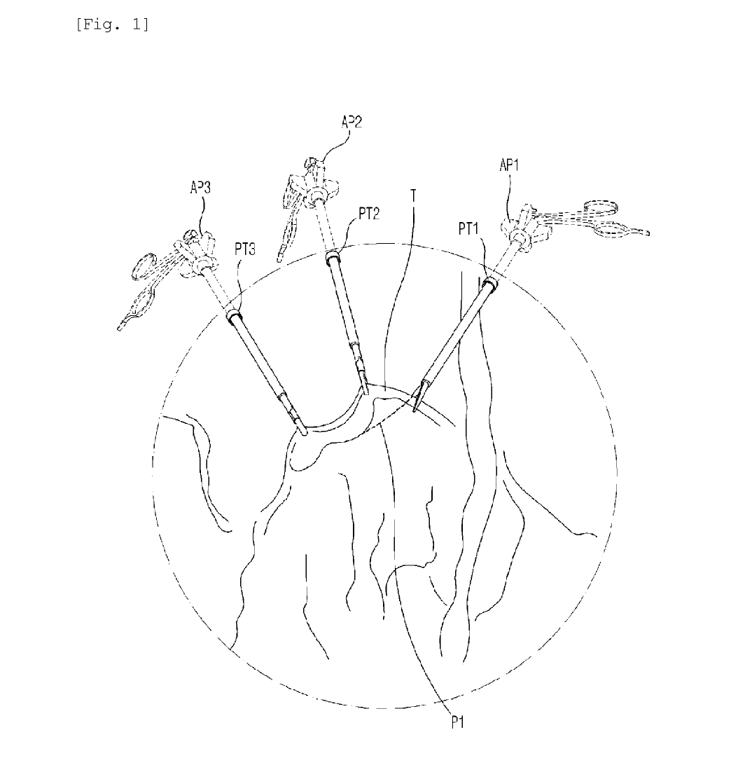

### MODE FOR INVENTION

The present invention may be embodied in many different forms without departing from the spirit and significant characteristics of the invention. Therefore, the embodiments of the present invention are disclosed only for illustrative purposes and should not be construed as limiting the present invention.

It will be understood that, although the terms first, second, etc. may be used herein to describe various elements, these elements should not be limited by these terms. These terms are only used to distinguish one element, from another element. For instance, a first element discussed below could be termed a second element without departing from the teachings of the present invention. Similarly, the second element could also be termed the first element. The term “and/or” includes any and all combinations of one or more of the associated listed items.

It will be understood that when an element is referred to as being “coupled” or “connected” to another element, it can be directly coupled or connected to the other element or intervening elements may be present therebetween. In contrast, it should be understood that when an element is referred to as being “directly coupled” or “directly connected” to another element, there are no intervening elements present.

The terminology used herein is for the purpose of describing particular embodiments only and is not intended to be limiting. As used herein, the singular forms are intended to include the plural forms as well, unless the context clearly indicates otherwise. It will be further understood that the terms “comprise,” “include,” “have,” etc., when used in this specification, specify the presence of stated features, integers, steps, operations, elements, components, or combinations thereof but do not preclude the presence or addition of one or more other features, integers, steps, operations, elements, components, and/or combinations thereof.

Unless otherwise defined, all terms including technical and scientific terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Hereinafter, preferred embodiments of the present invention will be described in detail with reference to the attached drawings. The same reference numerals will be used throughout the different drawings to designate the same or similar components, and the repetition of the same explanation for these components will be avoided. If, in the specification, detailed descriptions of well-known functions or configurations would unnecessarily obscure the gist of the present invention, the detailed descriptions will be omitted.

Hereinbelow, the construction of a surgical instrument according to the embodiment of the present invention will be described.

The surgical instrument according to an embodiment of the present invention is a surgical tool that is inserted into a body cavity S through a port PT1 so as to draw back an internal tissue T. The surgical instrument includes a mesh 100 and a surgical retraction means 200. Here, the internal tissue T means a part of the interior of the body which is an aggregate of cells and has a predetermined weight and volume. For example, the internal tissue may be an organ, a part of an organ, or a variety of body tissues having shapes, weights and volumes similar to those of organs.

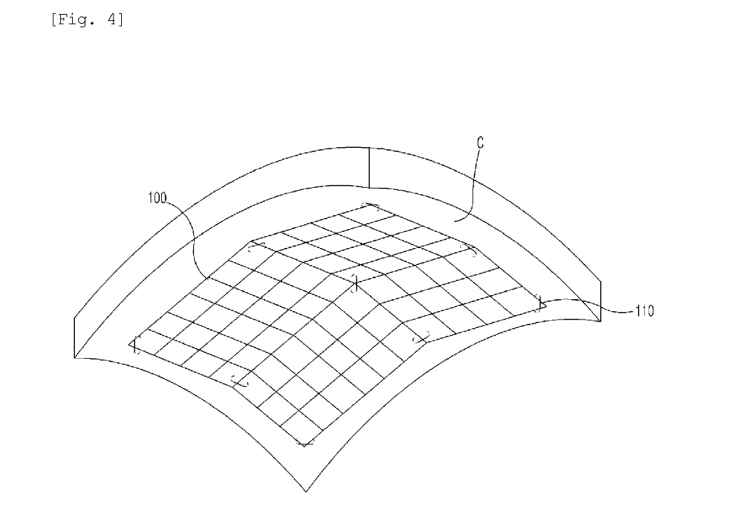

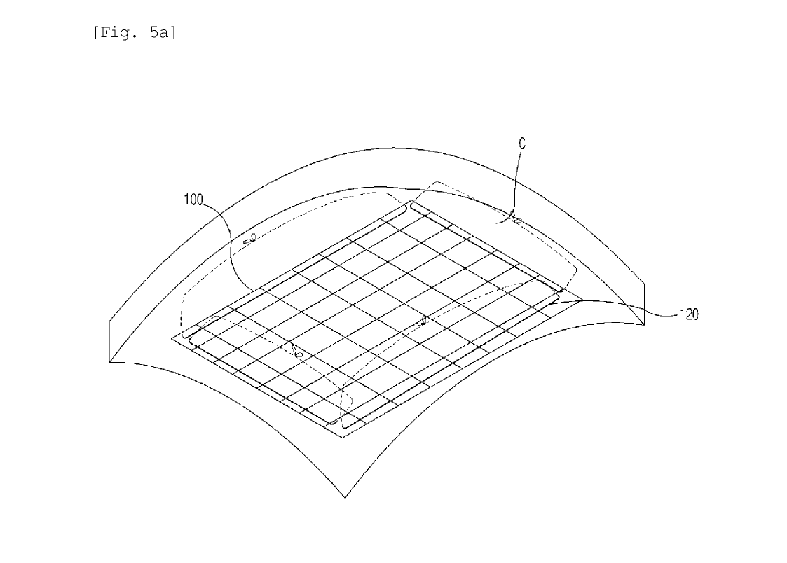

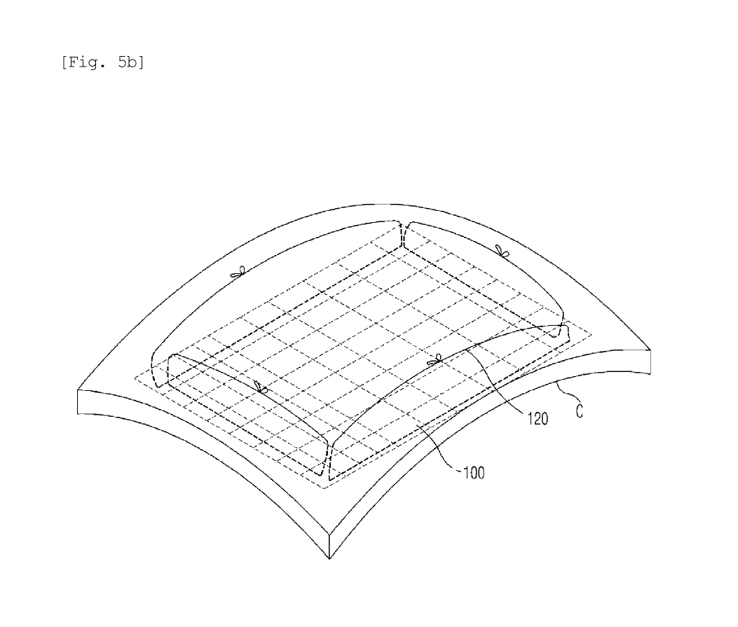

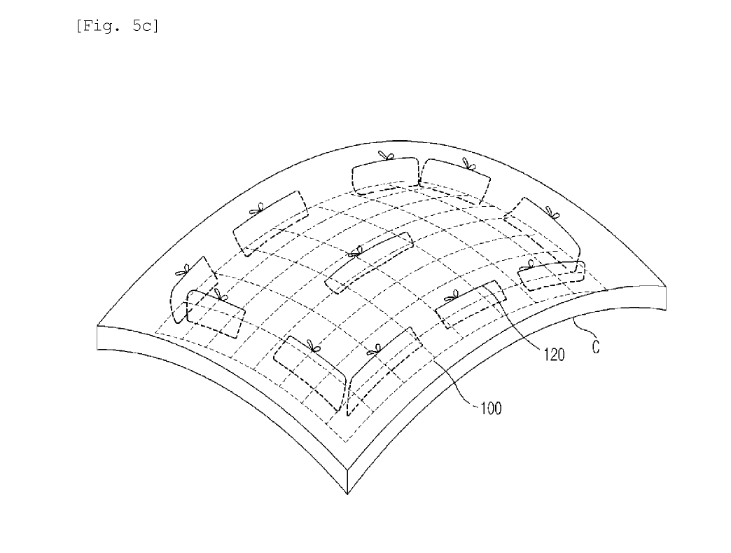

The mesh 100 is fastened in an open state to an inner surface C of the body cavity S by a fastening means 110 (FIG. 4) or 120 (FIG. 5a).

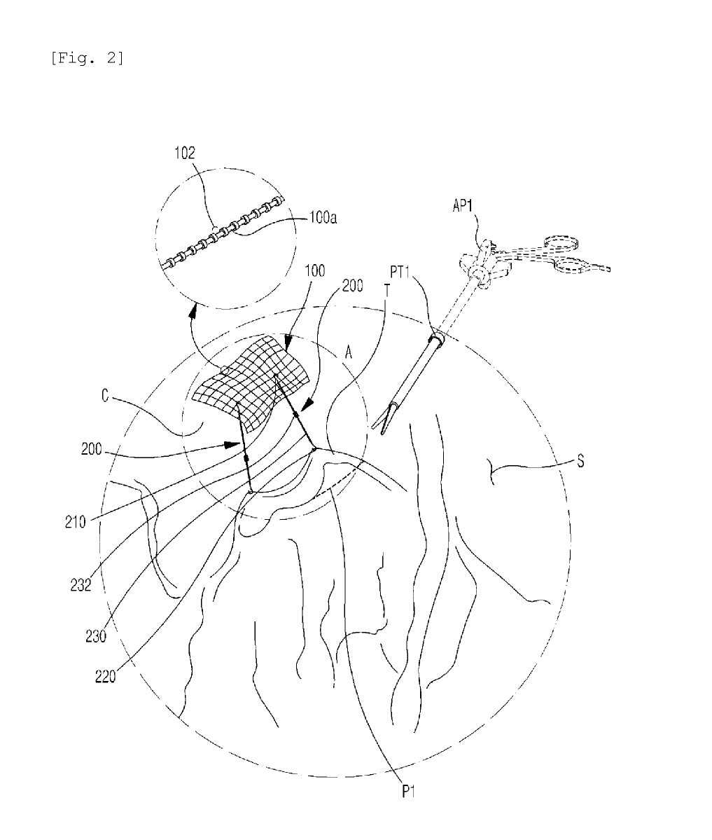

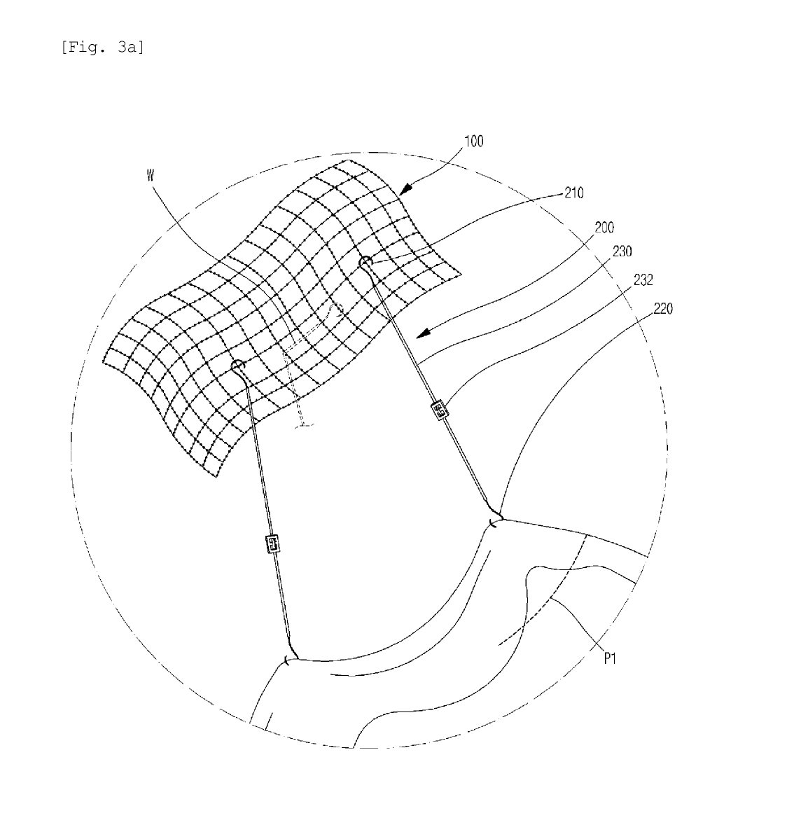

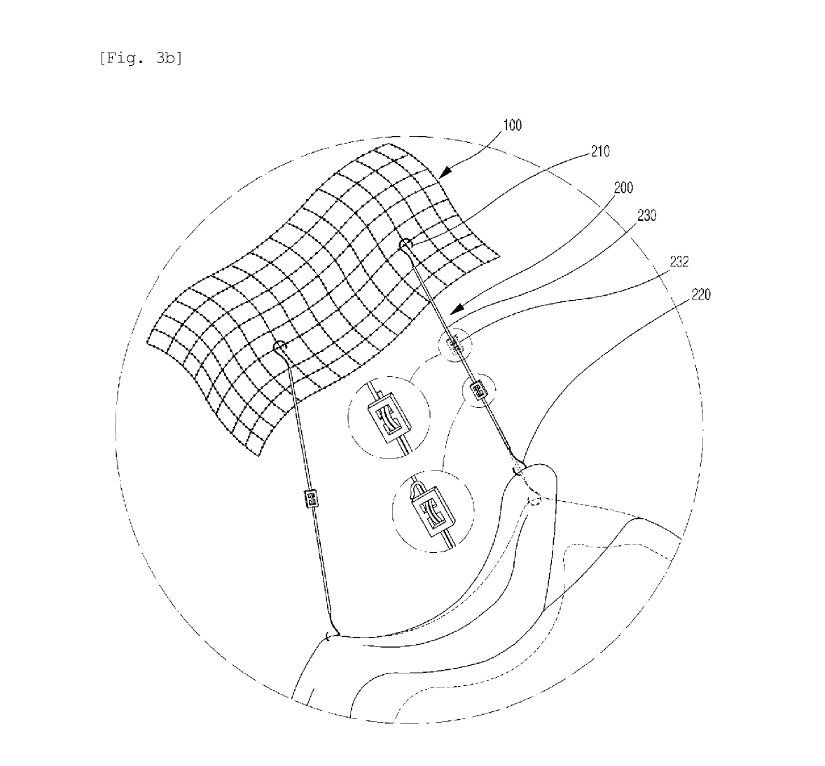

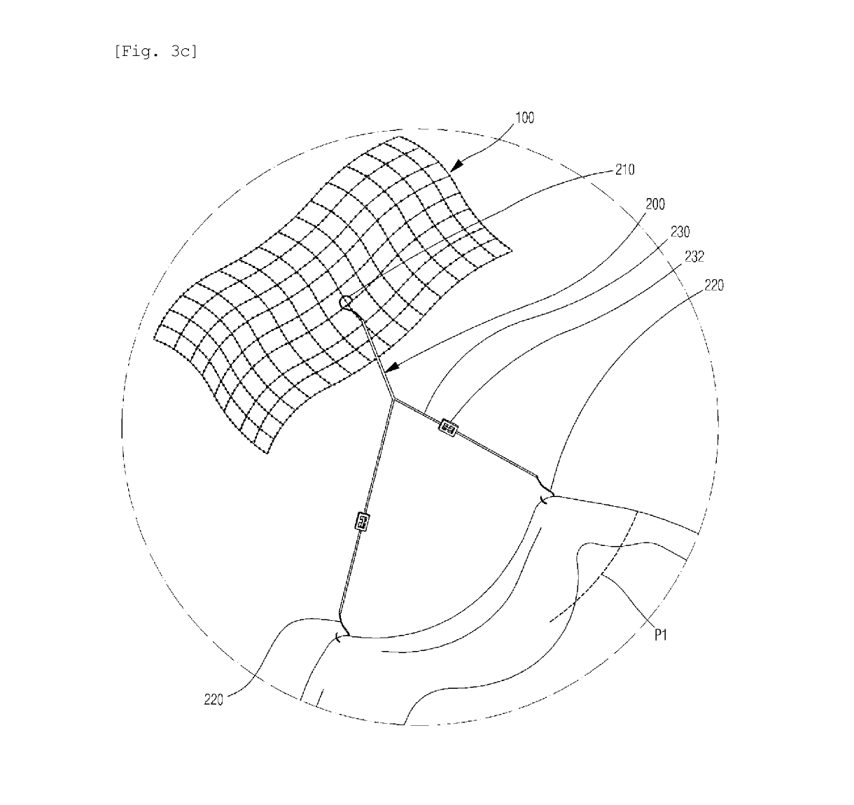

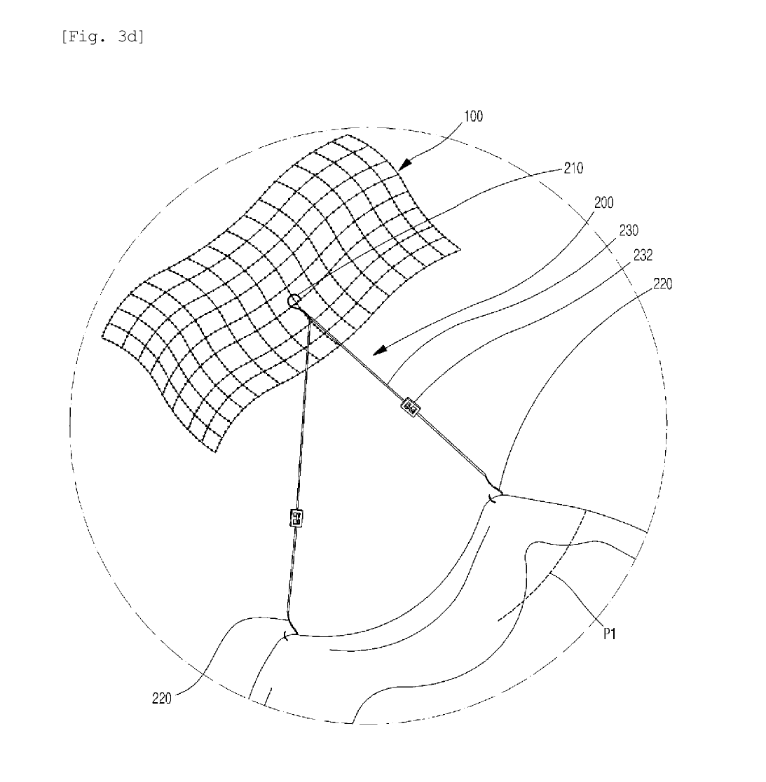

As shown in FIG. 2 to FIG. 3a, the mesh 100 may be formed by interlacing a plurality of threads 100a, in which grooves 102 may be formed on the surface of each thread 100a at predetermined intervals.

The grooves 102 function to restrict a mesh holding anchor 210 of the surgical retraction means 200 to a fastened position in the mesh 100.

In other words, when the mesh holding anchor 210 is fastened to one of the grooves 102 that are defined between neighboring protrusions, the mesh holding anchor 210 can be held in the fastened position by the groove 102.

To enable the grooves 102 to hold the mesh holding anchor 210 in the fastened state without moving, it is preferred that the grooves 102 be formed so as to have a thickness almost equal to that of the mesh holding anchor 210.

In other words, to form the grooves 102 on the surface of each thread 100a at predetermined intervals in such a way that the mesh holding anchor 210 can be fastened to one of the grooves 102, the protrusions are formed on the surface of each thread 100a at predetermined intervals.

The mesh 100 may be formed of a flexible material so that the shape of the mesh 100 can be freely deformed.

Particularly, it is preferred that the mesh 100 be made of an elastic and flexible material so that the mesh 100 can be elastically opened after the mesh 100 is inserted into the body cavity through the port PT1 in a rolled state.

The material of the mesh which can realize the above-mentioned function may be selected from synthetic resin materials, for example, PE, etc.

Further, the size of each opening between the interlacing threads of the mesh 100 may be determined to be a size, for example, 2 cm×2 cm, that allows an applier 300 or other surgical tools to pass through the opening.

Further, the size of each opening between the interlacing threads of the mesh 100 may be determined to be a smaller size, for example, 1 mm˜3 mm.

When the openings between the interlacing threads of the mesh 100 are small-sized as described above, the mesh holding anchor 210 can be efficiently held in the fastened state in the mesh 100 without moving even when no grooves 102 are formed on the threads of the mesh.

The fastening means for fastening the mesh 100 may comprise at least one staple 110 or at least one fastening string 120.

For example, the fastening means may comprise a plurality of staples 110 that can fasten predetermined parts of the mesh 100 to the inner surface C of the body cavity S by stapling them together, as shown in FIG. 4.

The staples 110 are surgical staples that are used in human surgery and are preferably made of a material harmless to the human body.

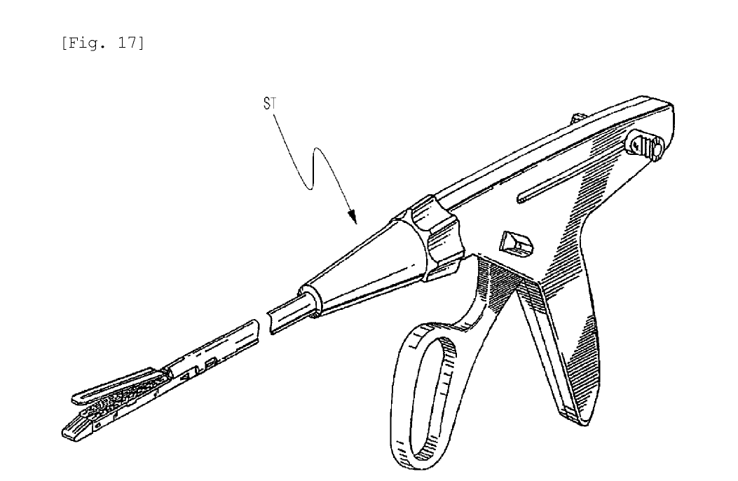

The staples 110 may be fastened to the inner surface C of the body cavity S using a conventional surgical stapler ST that is shown in FIG. 17.

Conventional techniques related to such surgical staplers may be referred to U.S. Pat. No. 8,056,788, etc.

In other words, the plurality of staples 110 fasten predetermined parts of the mesh 100 to the inner surface C of the body cavity S, and so the mesh 100 can be fastened to the inner surface C of the body cavity S.

Further, for example, the fastening means may comprise a plurality of fastening strings 120 that can fasten the mesh 100 to the inner surface C of the body cavity S by sewing with a needle in which the fastening strings 120 pass through predetermined openings of the mesh 100 and through predetermined parts of the inner surface C of the body cavity S, as shown in FIGS. 5a and 5b.

FIG. 5a illustrates an interior wall of the body, and FIG. 5b illustrates an exterior wall of the body.

In other words, a needle is inserted into the body cavity S from the exterior of the body, pierces through a desired part of the mesh 100 and, thereafter, passes to the exterior of the body prior to knotting, thereby fastening the mesh 100 to the inner surface C of the body cavity S.

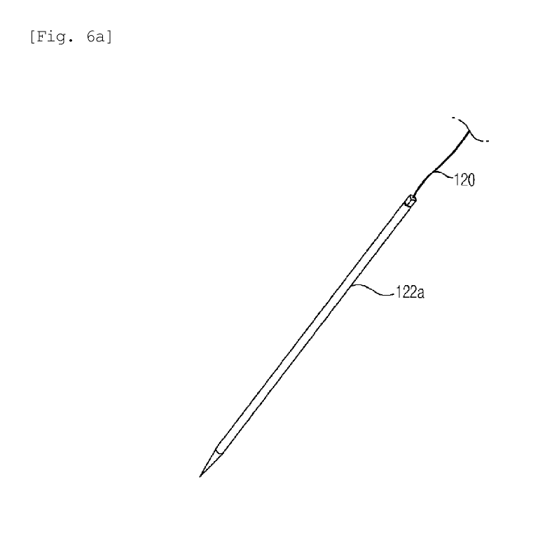

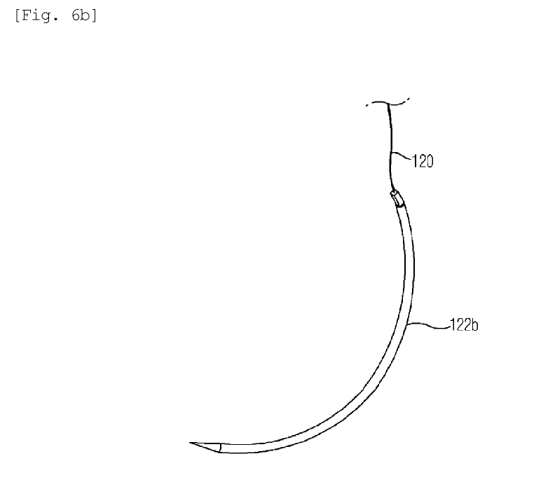

Here, the needle may be a straight needle 122a as shown in FIG. 6a, or a curved needle 122b as shown in FIG. 6b, and may be pertinently selected from the straight needle and the curved needle according to surgical conditions or the state of the abdominal walls.

Further, the number and positions of the knots of the fastening strings 120 may be pertinently changed according to the shape and size of the mesh 100 and/or according to a required retraction force.

For example, as shown in FIG. 5c, the fastening strings 120 may fasten the mesh 100 by being knotted at every predetermined positions.

Further, it is of course that the fastening means may comprise a combination of the staples 110 and the fastening strings 120.

Further, although it is not illustrated in the accompanying drawings, conventional clips that are used to fasten the tissue during laparoscopic surgery can be used when necessary.

Further, when fastening the mesh 100, the mesh 100 may be fastened to the inner surface C of the body cavity S with a predetermined interval remaining between them.

Here, the interval allows the mesh holding anchor 210 to be efficiently connected to the mesh 100 without interfering with the inner surface C of the body cavity S.

Further, to hold the mesh 100 so that the mesh 100 does not move in a state in which the surgical retraction means 200 draws back the tissue, it is preferred that the mesh 100 be fastened in a fully stretched state.

As shown in FIGS. 3a and 3b, the surgical retraction means 200 is removably connected to a predetermined point of the mesh 100 at a first end thereof. Further, a second end of the surgical retraction means 200 is connected to a predetermined point of the internal tissue T that is required to be drawn back, thereby drawing back the internal tissue T.

The surgical retraction means 200 comprises a mesh holding anchor 210, a tissue holding anchor 220 and a retraction string 230.

The mesh holding anchor 210 is selectively connected to a predetermined point of the mesh 100, and the tissue holding anchor 220 is connected to a predetermined point of the internal tissue T that is required to be drawn back.

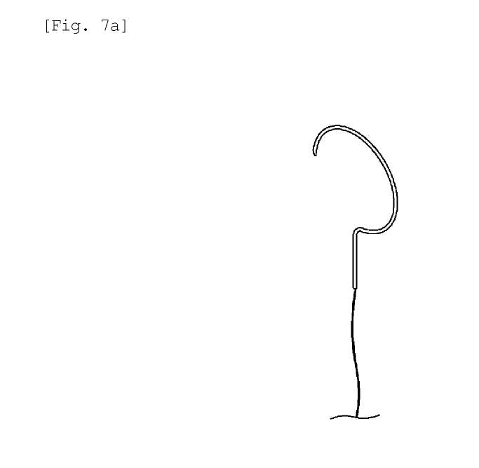

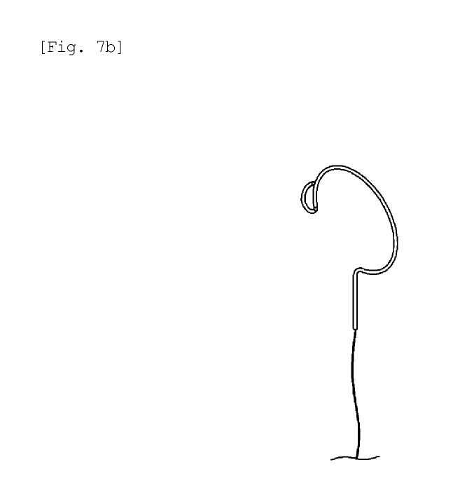

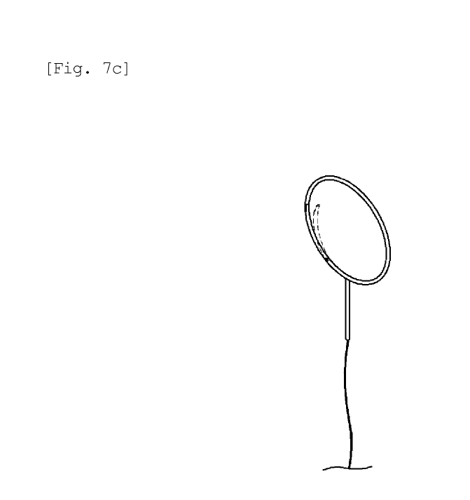

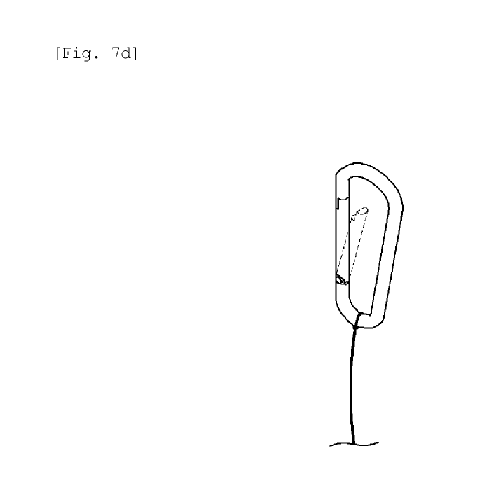

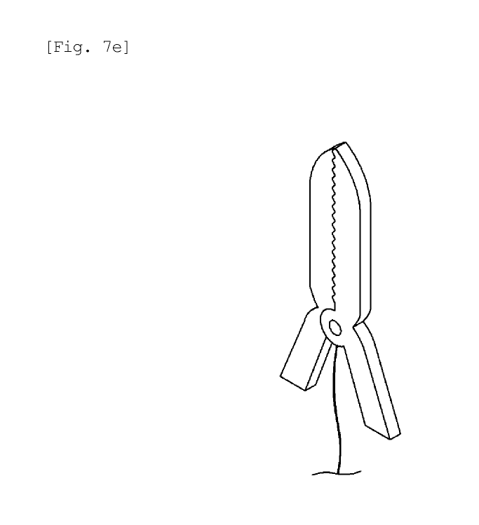

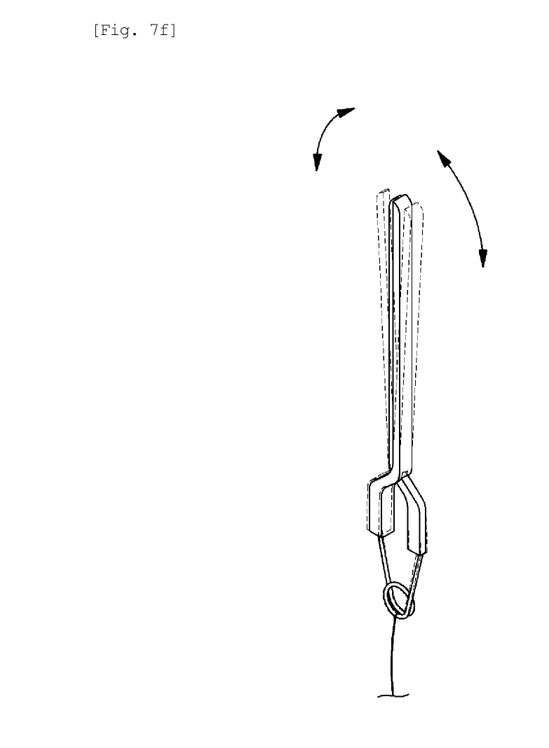

Each of the mesh holding anchor 210 and the tissue holding anchor 220 may be selected from an open hook type anchor as shown in FIGS. 7a and 7b, a snap hook type anchor as shown in FIG. 7c, a carabiner type anchor as shown in FIG. 7d, and a tongs type anchor as shown in FIGS. 7e and 7f.

For example, as shown in FIG. 7a, the mesh holding anchor 210 may be formed as an open hook type anchor that is open in a predetermined part, and so the mesh holding anchor 210 can be connected to a predetermined point of the mesh 100 by catching the point of the mesh 100.

Here, the end of the open hook type anchor may be roundly bent as shown in FIG. 7b, and so when the open hook type anchor is connected to the point of the mesh 100, the anchor can be prevented from causing damage to the inner surface C of the body cavity S.

Further, as shown in FIGS. 7c and 7d, the mesh holding anchor 210 may be formed as a snap hook type anchor (FIG. 7c) or a carabiner type anchor (FIG. 7d), in which the open part of each anchor can be elastically closed. In this case, to connect the anchor to the mesh, the anchor is coupled to a point of the mesh 100 in a state in which the open part is opened and, thereafter, the open part of the anchor is elastically closed, thereby finishing the connection of the anchor to the point of the mesh.

Further, as shown in FIGS. 7e and 7f, the mesh holding anchor 210 may be formed as a tongs type anchor which can be connected to a point of the mesh 100 by gripping the point of the mesh 100 using the front end thereof.

FIG. 7e illustrates a mesh holding anchor that is formed as a typical tongs type anchor, and FIG. 7f illustrates a mesh holding anchor that is formed as a bulldog clip type anchor.

Although only the mesh holding anchors 210 have been described in the above description, it should be understood that the tissue holding anchor 220 may be configured and operated in the same manner as that described for the mesh holding anchors 210, but the anchors 210, 220 are different from each other in that the tissue holding anchor 220 is connected to the tissue T.

Further, when considering that the tissue holding anchor 220 is connected to the tissue T required to be drawn back, it is preferred that the tissue holding anchor 220 be formed as an open hook type anchor that can be efficiently connected to the tissue T by piercing, or a tongs type anchor that can be efficiently connected to the tissue T by gripping.

Particularly, when the tissue holding anchor 220 is formed as the tongs type anchor, the tissue holding anchor 220 is provided with a tongs opening degree main control part that can control the opening degree of the hinged arms of the anchor.

Further, a tongs opening degree sub-control part may be provided on at least one side surface of the tongs opening degree main control part in such a way that the tongs opening degree sub-control part extends on the tongs opening degree main control part. This tongs opening degree sub-control part is used to subsidiarily control the opening degree of the hinged arms of the anchor.

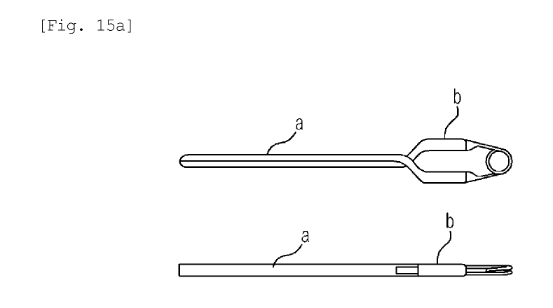

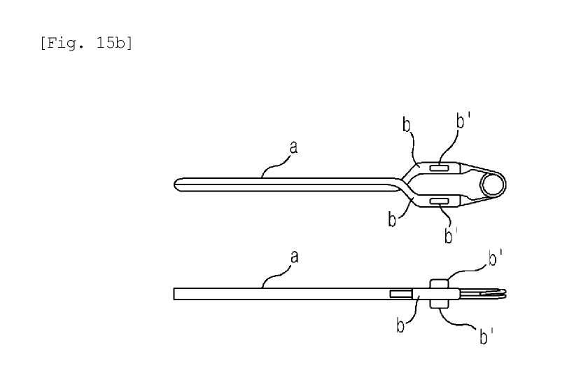

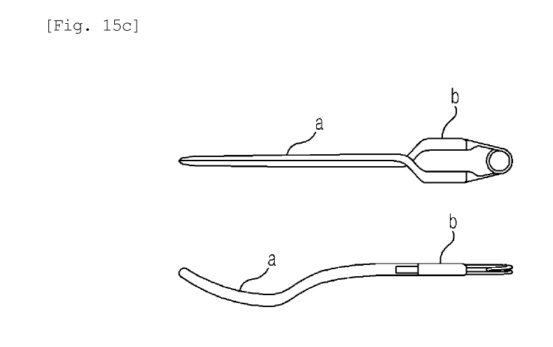

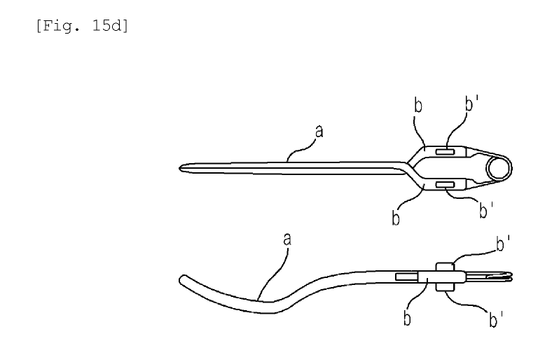

The tongs opening degree main control part and the tongs opening degree sub-control part will be described hereinbelow with reference to FIG. 15a to FIG. 15d.

As shown in FIG. 7f, the bulldog clip type mesh holding anchors are illustrated in detail in FIGS. 15a to 15d, in which FIGS. 15a and 15b illustrate straight bulldog clip anchors, and FIGS. 15c and 15d illustrate curved bulldog clip anchors.

Each of the bulldog clip anchors is provided with a tongs opening degree main control part b, as shown in FIGS. 15a and 15c, so as to control the opening degree of the hinged arms a of the anchor.

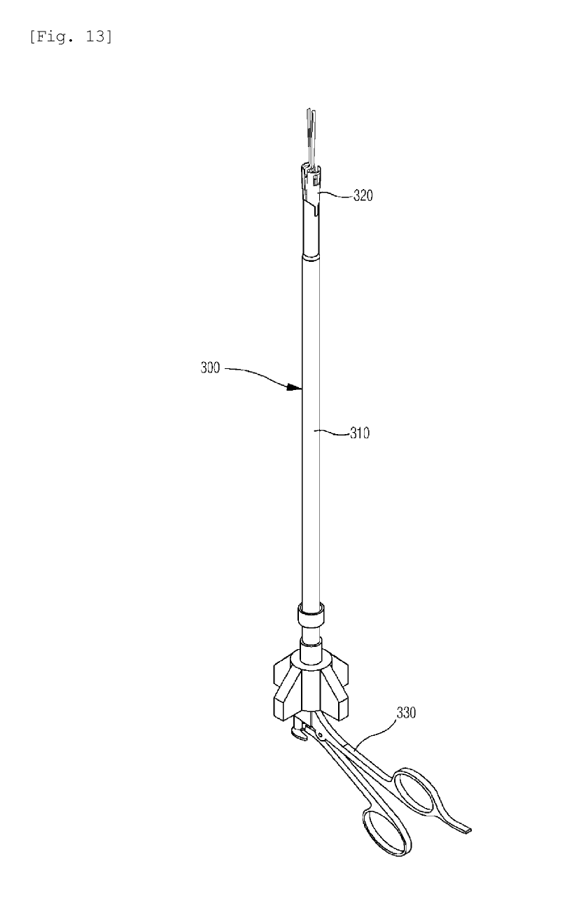

Accordingly, the opening degree of the hinged arms a of the anchor can be controlled by manipulating the tongs opening degree main control part b using an applier 300, as shown in FIG. 13.

As shown in FIGS. 15b and 15d, the bulldog clip anchor is further provided with a tongs opening degree sub-control part b′.

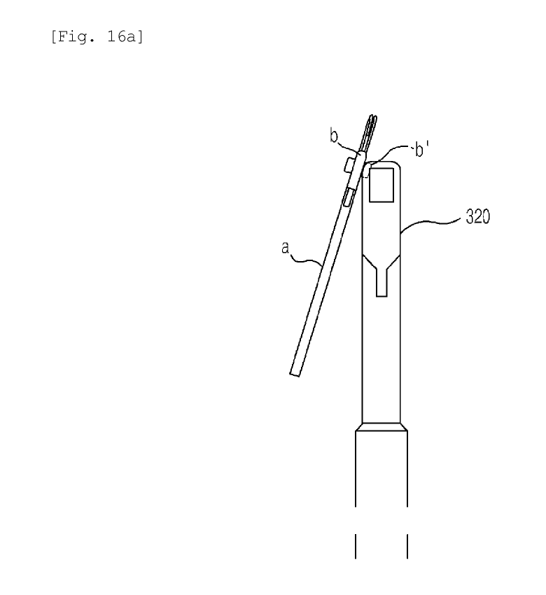

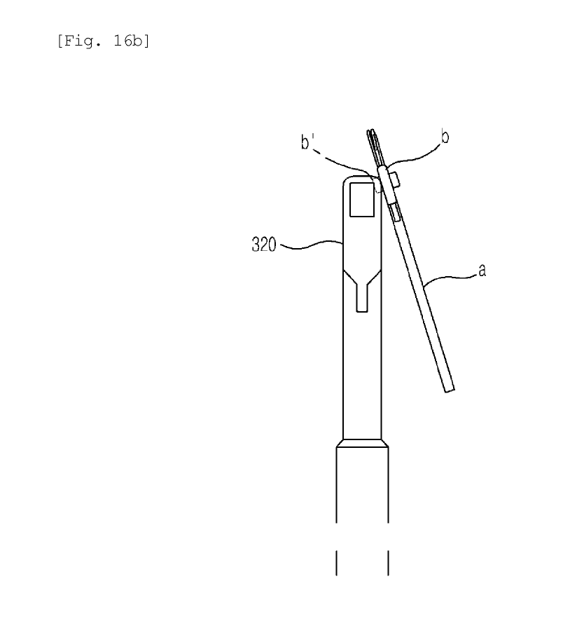

The tongs opening degree sub-control part b′ is a part that functions to subsidiarily control the opening degree of the hinged arms a of the anchor. As shown in FIGS. 16a and 16b, the opening degree of the hinged arms of the anchor can be controlled by manipulating the tongs opening degree sub-control part b′ using the applier 300.

Because the opening degree of the hinged arms a of the anchor can be controlled by manipulating the tongs opening degree sub-control part b′ that extends on the side surface of the tongs opening degree main control part b, the tissue gripping direction of the bulldog clip anchor can be freely controlled.

The retraction string 230 is connected to the mesh holding anchor 210 at the first end thereof and is connected to the tissue holding anchor 220 at the second end, thereby drawing back the internal tissue T.

As shown in FIG. 3b, the retraction string 230 may be formed as a band-shaped member having a predetermined width or may be formed as a linear member like a thread.

As shown in FIG. 7a to FIG. 7f, the retraction string 230 may be fastened to the mesh holding anchor 210 (or the tissue holding anchor 220) by being integrally connected thereto or by being tied thereto.

Further, to connect the retraction string 230 to the mesh holding anchor 210 (or the tissue holding anchor 220), the mesh holding anchor 210 (or the tissue holding anchor 220) is provided with a string connection part.

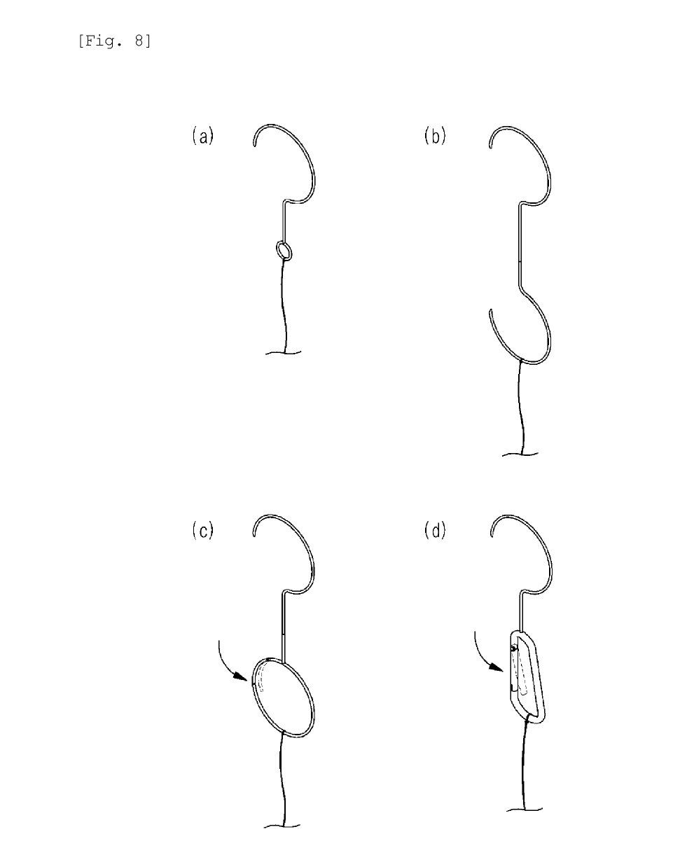

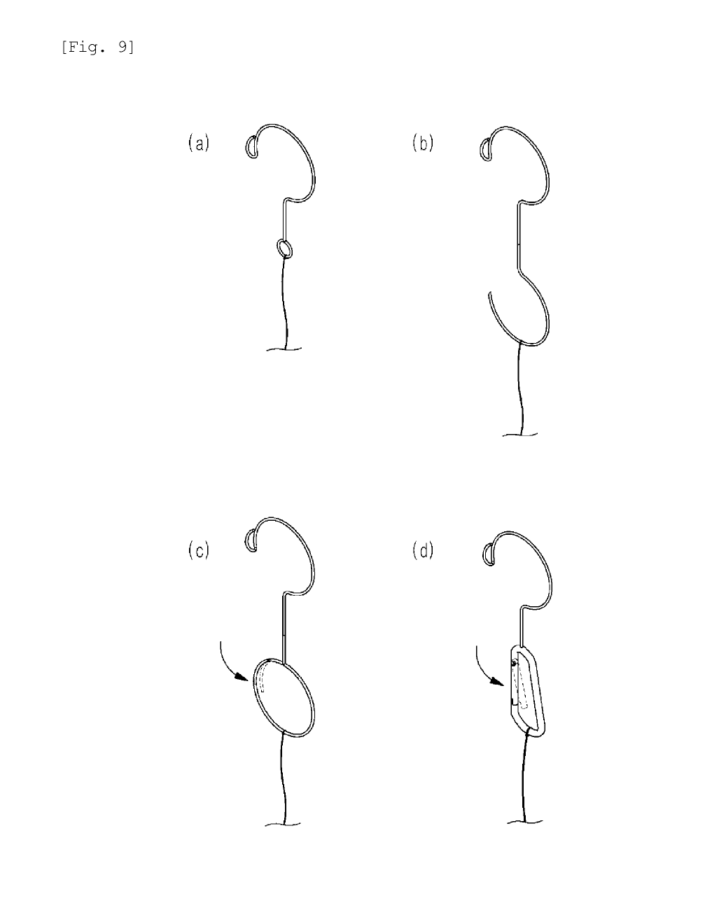

For example, as shown in FIGS. 8 and 9, the mesh holding anchor 210 may be formed as an open hook type, and the string connection part may be formed as any one of a ring type a, an open hook type b, a snap hook type c and a carabiner type d.

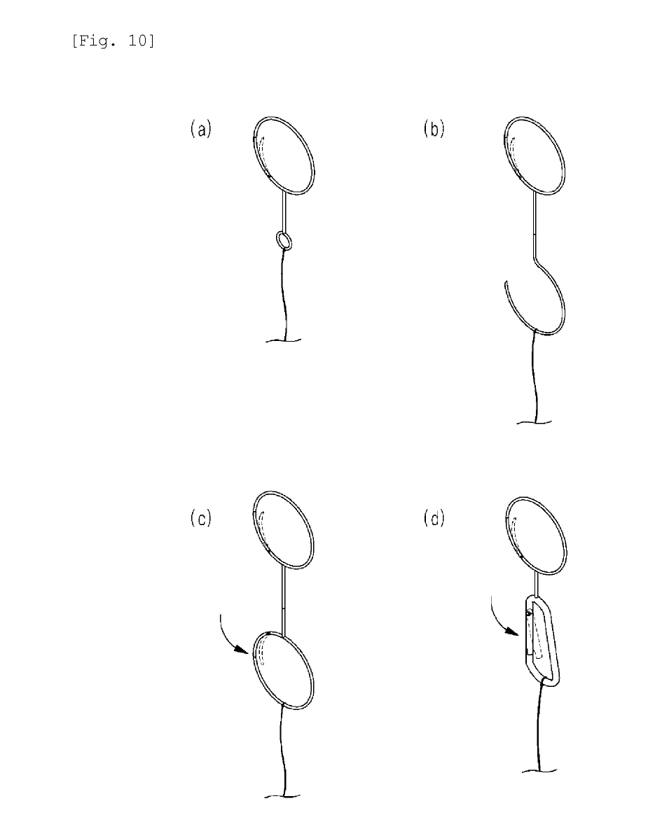

Further, as shown in FIG. 10, the mesh holding anchor 210 may be formed as a snap hook type, and the string connection part may be formed as any one of a ring type a, an open hook type b, a snap hook type c and a carabiner type d.

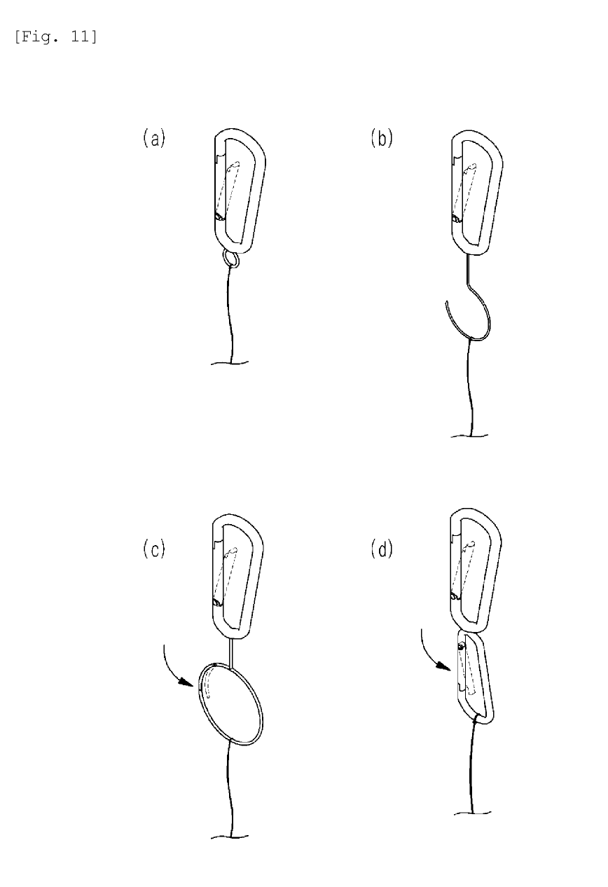

Further, as shown in FIG. 11, the mesh holding anchor 210 may be formed as a carabiner type, and the string connection part may be formed as any one of a ring type a, an open hook type b, a snap hook type c and the carabiner type d.

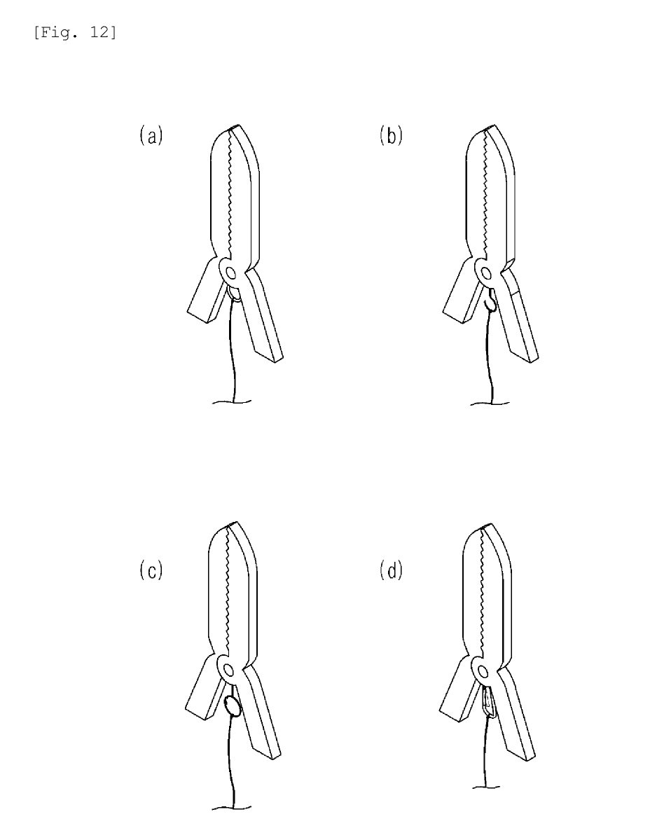

Further, as shown in FIG. 12, the mesh holding anchor 210 may be formed as a tongs type, and the string connection part may be formed as any one of a ring type a, an open hook type b, a snap hook type c and the carabiner type d.

When the string connection part is provided in the mesh holding anchor 210 (or the tissue holding anchor 220) as described above, the connection of the retraction string 230 to the anchor may be realized by tying the string to the string connection part.

Here, the retraction string 230 may be configured to have electricity.

Accordingly, even when the state of the tissue T that is being drawn back by the retraction string 230 and the retraction load of the retraction string 230 are changed, the retraction string 230 can continuously provide the desired retraction force. This function of the retraction string 230 will be described in detail in the description for a surgical method of the present invention.

The retraction string 230 may be provided with a length control means 232, and so the retracting position of the tissue T that is being drawn back by the retraction string 230 can be appropriately displaced to a desired position.

The length control means 232 may be configured as shown in FIG. 3b. Alternatively, the length control means 232 may be formed using a conventional length control means, such as a cable tie using a ratchet mechanism, which can control the length of a linear member.

In the above description, the connection of the anchors has been described in which one mesh holding anchor 210 and one tissue holding anchor 220 are connected to each other in a one to one method using one retraction string 230. However, it should be understood that one mesh holding anchor 210 may be connected to a plurality of tissue holding anchors 220, as shown in FIG. 3c, and the very reverse case may be possible.

In other words, a plurality of retraction strings 230 that are connected to a plurality of tissue holding anchors 220 may be gathered by knotting, and one of the retraction strings 230 may extend from the knot to the mesh holding anchor 210 so as to be connected thereto.

Further, as shown in FIG. 3d, the plurality of retraction strings 230 that are connected to the plurality of tissue holding anchors 220 may extend to the mesh holding anchor 210 so as to be commonly connected thereto.

Further, the surgical instrument according to the embodiment of the present invention may comprise the mesh 100 that is inserted into the body cavity S and the applier 300 that is used to manipulate the surgical retraction means 200.

The applier 300 comprises a shaft 310, a predetermined part of which is inserted into the body cavity S through the port PT1, a manipulation part 320 that is provided in the first end of the shaft 310, and a handle part 330 that is provided in the second end of the shaft 310 and controls the manipulation part 320.

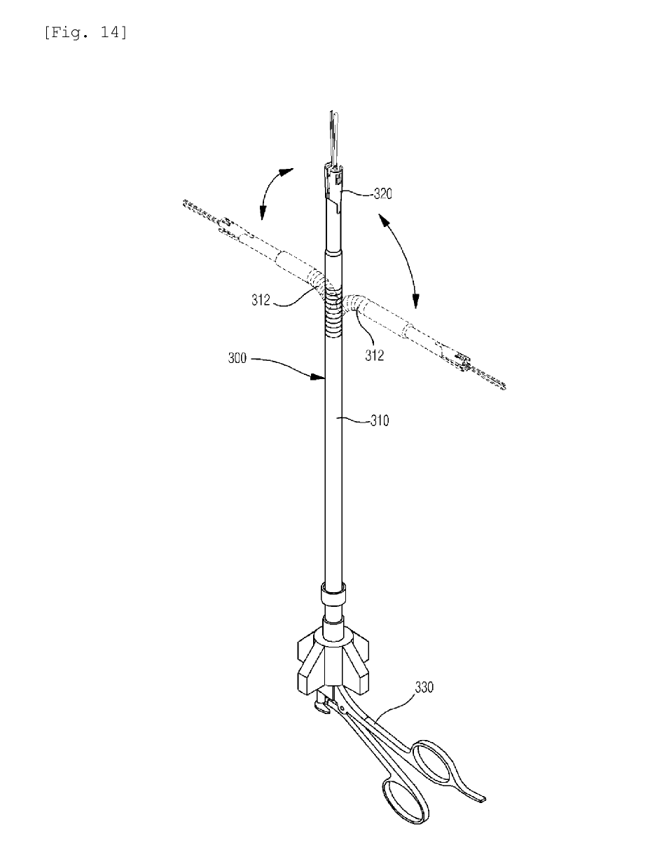

Further, as shown in FIG. 14, a joint 312 may be provided on the predetermined part of the shaft 310 such that the joint 312 can be controlled by the handle part 330 so as to change the manipulation range of the manipulation part 320.

The above-mentioned joint 312 is well-known to those skilled in the art related to the applier 300 and further explanation for the construction and operation of the joint 312 will be omitted in this description.

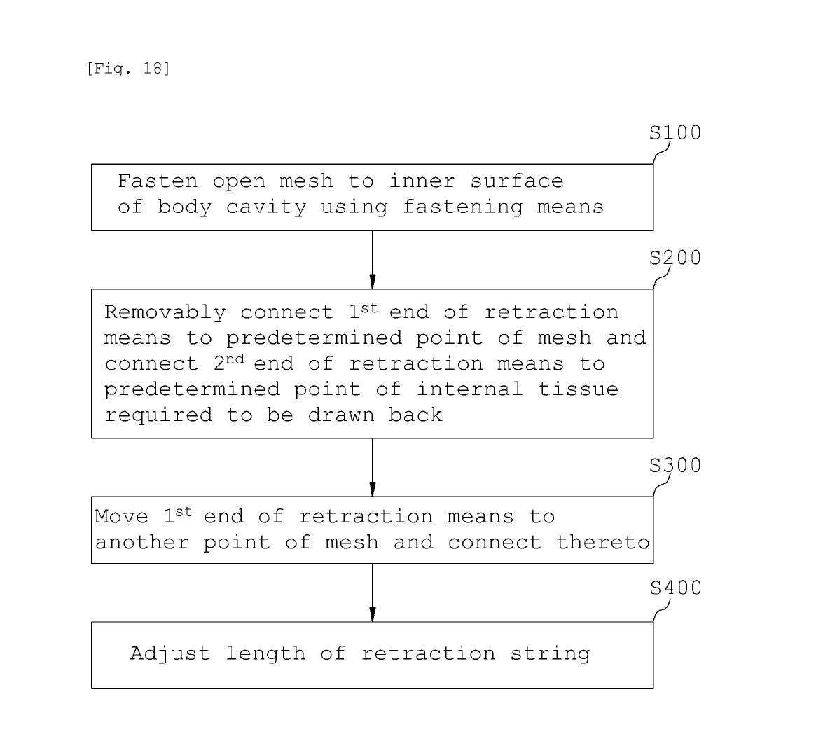

Hereinbelow, a surgical method performed using the above-mentioned surgical instrument will be described with reference to FIG. 18.

The surgical method according to the embodiment of the present invention is a surgical operative method in which the mesh 100 and the surgical retraction means 200 of the surgical instrument are inserted into the body cavity S through the port PT1 so as to draw back the internal tissue T.

First, the mesh 100 in an open state is fastened to the inner surface C of the body cavity S using the fastening means (S100).

The mesh 100 can be inserted into the body cavity S through the port PT1. Here, for example, the mesh 100 may be inserted into the body cavity S after being rolled to form a cylindrical shape.

After inserting the rolled mesh 100 into the body cavity S, the rolled mesh 100 is opened and is placed on the inner surface C of the body cavity S, and is fastened to the inner surface C of the body cavity S using the fastening means.

Here, the fastening means may comprise at least one staple 110 or at least one fastening string 120.

For example, the fastening of the mesh 100 to the inner surface C of the body cavity S may be realized using a plurality of staples 110 that can fasten predetermined parts of the mesh 100 to predetermined parts of the inner surface C of the body cavity S by stapling them together.

Further, for example, the fastening of the mesh 100 to the inner surface C of the body cavity S may be realized using a fastening string 120 that can fasten the mesh 100 to the inner surface C of the body cavity S by sewing with a needle in which the fastening string 120 passes through predetermined openings of the mesh 100 and through predetermined parts of the inner surface C of the body cavity S.

Thereafter, the first end of the surgical retraction means 200 is removably connected to a predetermined point of the mesh 100 and the second end of the surgical retraction means 200 is connected to a predetermined point of the internal tissue T that is required to be drawn back, thereby drawing back the internal tissue T (S200)

Here, conversely, the first end of the surgical retraction means 200 may be primarily connected to the predetermined point of the internal tissue T that is required to be drawn back, and the second end of the surgical retraction means 200 may be secondarily and removably connected to the predetermined point of the mesh 100.

For example, the surgical retraction means 200 may comprise a mesh holding anchor 210, a tissue holding anchor 220 and a retraction string 230, and the detailed construction of the parts of the surgical retraction means 200 may be referred to the above-mentioned description.

For example, when the surgical retraction means has an integrated structure in which the mesh holding anchor 210, the tissue holding anchor 220 and the retraction string 230 are integrated into a single body, the surgical retraction means can drawn back the tissue T by the following procedure.

1) The tissue holding anchor 220 is connected to the tissue T that is required to be drawn back.

2) The mesh holding anchor 210 is removably connected to a predetermined point of the mesh 100.

By the above-mentioned procedure, the retraction string 230 that connects the mesh holding anchor 210 and the tissue holding anchor 220 together can draw back the tissue T.

Further, for example, when the surgical retraction means has a structure in which the mesh holding anchor 210 and the tissue holding anchor 220 have respective string connection parts and opposite ends of the retraction string 230 are tied to the respective string connection parts, the surgical retraction means can drawn back the tissue T by the following procedure.

1) The tissue holding anchor 220 is connected to the tissue T that is required to be drawn back.

2) The mesh holding anchor 210 is removably connected to a predetermined point of the mesh 100.

3) Connecting opposite ends of the retraction string 230 to the respective string connection parts in a state in which the length of the retraction string 230 is controlled so as to efficiently draw back the tissue T.

By the above-mentioned procedure, the retraction string 230 that connects the mesh holding anchor 210 and the tissue holding anchor 220 together can draw back the tissue T.

Hereinbelow, a method of connecting a tongs type tissue holding anchor 220 that is shown in FIGS. 15a and 15b and is one of the various types of tissue holding anchors 220 to the tissue T that is required to be drawn back, in which the connection of the anchor 220 to the tissue T should be performed to realize a desired directional connection, will be described.

To control the opening degree of the hinged arms of the tongs type tissue holding anchor 220, both a tongs opening degree main control part b and a tongs opening degree sub-control part b′ that is formed on at least one side surface of the tongs opening degree main control part b are provided in the anchor 220.

Here, the tongs type tissue holding anchor 220 may be manipulated to grip the tissue T by controlling the opening degree of the hinged arms a of the anchor 220 using a straight applier 300 shown in FIG. 13 in a state in which the anchor 220 is positioned such that the direction of the hinged arms a of the anchor 220 is aligned with the axial direction of the shaft 310 of the applier 300.

Further, when using an applier 300 having a joint 312 as shown in FIG. 14, the tissue gripping direction of the hinged arms a of the tongs type tissue holding anchor 220 may be controlled by controlling the bending degree of the joint 312 of the applier 300 in a state in which the anchor 220 is positioned such that the direction of the hinged arms a of the anchor 220 is aligned with the axial direction of the shaft 310 of the applier 300.

Further, the tongs type tissue holding anchor 220 may be manipulated to grip the tissue T by controlling the hinged arms a of the anchor 220 using the straight applier 300 shown in FIGS. 16a and 16b in such a way that the manipulation part 320 of the applier 300 manipulates the tongs opening degree sub-control part b′ in a state in which the hinged arms a of the anchor 220 are directed in a desired direction.

Further, when it is required to change the retracting position of the tissue T according to a change in the surgical conditions, the first end of the surgical retraction means 200 can be moved to another point of the mesh 100 and can be connected thereto (S300).

The selective movement and connection of the first end of the surgical retraction means 200 to another point of the mesh 100 may be realized by moving the connecting position of the mesh holding anchor 210 to the other point of the mesh 100 and by connecting the mesh holding anchor 210 to the other point of the mesh 100.

Further, when it is required to change the retracting length of the tissue T according to a change in the surgical conditions, the length of the retraction string 230 may be adjusted (S400).

The adjustment in the length of the retraction string 230 may be realized by a manipulation of the length control means 232 of the retraction string 230.

Further, the adjustment in the length of the retraction string 230 may be realized by moving the connecting position of the mesh holding anchor 210 to another point of the mesh 100 in a state in which the retraction string 230 is suspended on the interlaced threads 100a of the mesh 100, as shown by the part W of FIG. 3a.

Further, the retraction string 230 may be configured to have elasticity.

For example, when the state of the tissue T is changed according to an excision of a specified part P1 of the tissue T as shown in FIGS. 3a and 3b (the weight of the tissue T that is being drawn back by the retraction string 230 is changed), the tissue T is further drawn back by the elasticity of the retraction string 230, and so the excised part is more widely open to view.

Further, the staples 110 that are installed in the inner surface C of the body cavity S are made of a material harmless to the human body, and so it is possible to leave the harmless staples 110 in the body cavity S by removing only the mesh 100 by cutting parts of the mesh 100 which correspond to the staples 110 after finishing surgery.

Further, it is obvious, of course, that the staples 110 may be removed from the inner surface C of the body cavity S using a separate staple remover means (not shown).

Although the preferred embodiments of the present invention have been disclosed for illustrative purposes, those skilled in the art will appreciate that various modifications, additions and substitutions are possible, without departing from the scope and spirit of the invention. Accordingly, the bounds of the present invention must be interpreted by the accompanying claims which can include all the modifications, additions and substitutions.

1. A surgical instrument configured to be inserted and installed in a body cavity so as to draw back an internal tissue, the surgical instrument comprising:
a mesh configured to be fastened in an open state to an inner surface of the body cavity using a fastening means; and
a surgical retraction means configured to be selectively and removably connected at a first end thereof to a predetermined point of the mesh and configured to be connected at a second end thereof to a predetermined part of the internal tissue that is required to be drawn back, so that the surgical retraction means can draw back the internal tissue.

2. The surgical instrument as set forth in claim 1, wherein the surgical retraction means comprises:
a mesh holding anchor configured to be selectively connected to the predetermined point of the mesh;
a tissue holding anchor configured to be connected to the predetermined part of the internal tissue that is required to be drawn back; and
a retraction string connected at a first end thereof to the mesh holding anchor and connected at a second end thereof to the tissue holding anchor.

3. The surgical instrument as set forth in claim 1, wherein a thread that constitutes the mesh is provided on a surface thereof with a plurality of grooves for firmly connecting the surgical retraction means to the mesh.

4. The surgical instrument as set forth in claim 1, wherein the fastening means comprises at least one staple that can fasten a predetermined part of the mesh to a predetermined part of the inner surface of the body cavity by stapling them together.

5. The surgical instrument as set forth in claim 1, wherein the fastening means comprises at least one fastening string that can fasten the mesh to the inner surface of the body cavity by sewing with a needle in which the fastening string passes through predetermined openings of the mesh and through predetermined parts of the inner surface of the body cavity.

6. The surgical instrument as set forth in claim 5, wherein the needle is configured as a straight needle or a curved needle.

7. The surgical instrument as set forth in claim 2, wherein the mesh holding anchor or the tissue holding anchor is configured as any one of an open hook type anchor, a snap hook type anchor, a carabiner type anchor and a tongs type anchor.

8. The surgical instrument as set forth in claim 7, wherein the tissue holding anchor is configured as the tongs type anchor provided with a tongs opening degree main control part, with a tongs opening degree sub-control part being formed on at least one side surface of the tongs opening degree main control part.

9. The surgical instrument as set forth in claim 2, wherein the mesh holding anchor or the tissue holding anchor is provided with a string connection part that is configured as any one of a ring type, an open hook type, a snap hook type and a carabiner type, thereby being connected to the retraction string.

10. The surgical instrument as set forth in claim 2, wherein the retraction string is configured to have elasticity.

11. The surgical instrument as set forth in claim 2, wherein the retraction string is provided with a length control means.

12. The surgical instrument as set forth in claim 1, further comprising:
an applier comprising a shaft, a predetermined part of which is inserted into the body cavity through a port, a manipulation part provided in a first end of the shaft, and a handle part provided in a second end of the shaft so as to control the manipulation part, so that the applier can manipulate the mesh and the surgical retraction means that are inserted in the body cavity.

13. The surgical instrument as set forth in claim 12, wherein the shaft is provided in the predetermined part thereof with a joint that can be controlled by the handle part so that a manipulation range of the manipulation part can be changed by the joint.

14. A surgical mesh configured to be inserted and installed in a body cavity so as to draw back an internal tissue, the surgical mesh being configured to be fastened in an open state to an inner surface of the body cavity using a fastening means.

15. The surgical mesh as set forth in claim 14, wherein a thread that constitutes the mesh is provided on a surface thereof with a plurality of grooves for firmly connecting a surgical retraction means to the mesh.

16. A surgical retraction means configured to be inserted into a body cavity so as to draw back an internal tissue, the surgical retraction means comprising:
a mesh holding anchor configured to be selectively connected to a predetermined point of the mesh that is fastened to an inner surface of the body cavity;
a tissue holding anchor configured to be connected to a predetermined part of the internal tissue that is required to be drawn back; and
a retraction string connected at a first end thereof to the mesh holding anchor and connected at a second end thereof to the tissue holding anchor.

17. The surgical retraction means as set forth in claim 16, wherein the mesh holding anchor or the tissue holding anchor is configured as any one of an open hook type anchor, a snap hook type anchor, a carabiner type anchor and a tongs type anchor.

18. The surgical retraction means as set forth in claim 17, wherein the tissue holding anchor is configured as the tongs type anchor provided with a tongs opening degree main control part, with a tongs opening degree sub-control part being formed on at least one side surface of the tongs opening degree main control part.

19. The surgical retraction means as set forth in claim 16, wherein the mesh holding anchor or the tissue holding anchor is provided with a string connection part that is configured as any one of a ring type, an open hook type, a snap hook type and a carabiner type, thereby being connected to the retraction string.

20. The surgical retraction means as set forth in claim 16, wherein the retraction string is configured to have elasticity.

21. The surgical retraction means as set forth in claim 16, wherein the retraction string is provided with a length control means.

22. A surgical method performed in a state in which a surgical instrument is inserted into a body cavity through a port and draws back an internal tissue, the surgical method comprising:
fastening a mesh in an open state to an inner surface of the body cavity using a fastening means; and
selectively and removably connecting a first end of a surgical retraction means to a predetermined point of the mesh and connecting a second end of the surgical retraction means to a predetermined part of the internal tissue that is required to be drawn back, thereby drawing back the internal tissue.

23. The surgical method as set forth in claim 22, further comprising:
moving and connecting the first end of the surgical retraction means to another point of the mesh.

24. The surgical method as set forth in claim 22, wherein the surgical retraction means comprises:
a mesh holding anchor selectively connected to the predetermined point of the mesh;
a tissue holding anchor connected to the predetermined part of the internal tissue that is required to be drawn back; and
a retraction string connected at a first end thereof to the mesh holding anchor and connected at a second end thereof to the tissue holding anchor, thereby drawing back the internal tissue.

25. The surgical method as set forth in claim 24, further comprising:
controlling a length of the retraction string.

26. The surgical method as set forth in claim 24, wherein the moving and connecting the first end of the surgical retraction means to the other point of the mesh is performed by moving a connecting position of the mesh holding anchor to the other point of the mesh and by connecting the mesh holding anchor to the other point of the mesh.
